(19) United States
(12) Patent Application Publication
(10) Pub. No.: US 2011/0118144 A1
(43) Pub. Date: May 19, 2011

(54) ENGINEERED PHAGE VECTORS FOR THE DESIGN AND THE GENERATION OF A HUMAN NON-ANTIBODY PEPTIDE OR PROTEIN PHAGE LIBRARY VIA FUSION TO PIX OF M13 PHAGE

(76) Inventors: Linus Hyun, Radnor, PA (US); Chichi Huang, Radnor, PA (US); Karyn O'Neil, Radnor, PA (US)

(21) Appl. No.: 12/743,246

(22) PCT Filed: Nov. 21, 2008
(86) PCT No.: PCT/US08/84317

§ 371 Date: May 17, 2010

Related U.S. Application Data
(60) Provisional application No. 61/014,777, filed on Dec. 19, 2007.

(51) Int. Cl. C40B 30/08; C12N 15/63; C12N 1/21; C07K 14/195; C40B 40/02

(52) U.S. Cl. 506/11; 435/320.1; 435/252.3; 530/350; 506/14

(57) ABSTRACT

The invention relates to a compositions and methods for generating and using pIX phage display libraries for producing non-antibody peptide or protein proteins or peptides using engineered hybrid phage vectors derived from pIX of M 13 phage.

### FIELD OF THE INVENTION

The invention relates to a compositions and methods for generating and using pIX phage display libraries for producing non-antibody peptide or protein proteins or peptides using engineered hybrid phage vectors derived from pIX of M13 phage.

### BACKGROUND OF THE INVENTION

Phage display is a well-established tool for affinity-based selection of polypeptides. In a typical phage display selection, a library of polypeptides is genetically fused to the terminus of one of the coat proteins of the filamentous phage M13. The phage particle provides a physical link between each polypeptide member of the library and the gene that encodes it. The phage library can then be affinity-selected, or panned, for those members of the library that bind to a desired target molecule. The library is mixed with the target, unbound phage particles are washed away, and the remaining phage eluted and amplified by culturing in E. coli cells.

Although the display of foreign polypeptides has been accomplished with each of the coat proteins of M13, pIII and pVIII are by far the most common fusion partners. pIII is a 42 kD minor coat protein that is responsible for phage infection into E. coli. Each phage particle contains up to five copies of the pIII protein on its surface, gathered at one end of the phage. PVIII is the major coat protein of the phage; thousands of copies of pVIII (molecular weight 5 kD) are arranged in an orderly fashion around the single-stranded viral genome to comprise the phage capsid. In addition to pIII, M13 has three other minor coat proteins: pVI, a 12 kD protein, and pVII and pIX, which are short proteins (33 and 32 amino acids, respectively) that are involved in initiation of assembly and maintenance of stability. Five copies of the pVI protein lie at the same end of the phage as pIII, while five copies each of pVII and pIX reside at the opposite end of the phage.

While phage libraries displaying fusions to pIII and pVIII have proven productive in many cases, the polypeptides displayed by phage are subject to certain biological constraints. For instance, most peptides of eight or more amino acids in length do not display well as fusions to pVIII. In addition, polypeptides that interact with the phage protein itself or otherwise affect the expression, incorporation, or activity of pIII or pVIII will be under-represented in the library, because the phage that display them will not grow well. Finally, because pIII is a rather large protein, access of pIII-displayed polypeptides to certain target sites (deep, narrow crevices on a protein surface, for instance), or the correct assembly of polypeptides that function best in multimeric form, might be sterically hindered. Thus, selections from phage libraries that exploit other coat proteins—which have different structures and biological functions and thus might be expected to impose different constraints on displayed polypeptides would help to ensure that a maximum amount of sequence diversity is searched. In proof of concept experiments, it has been shown that pVII and pIX can be used for the display of both antibody fragments and peptides. These results were especially noteworthy since earlier work had suggested that fusions of polypeptides to the N-termini of pVII and pIX render these coat proteins non-functional.

The display of foreign polypeptides on phage is accomplished through the use of phage, phagemid, or hybrid vectors. With phage vectors, the gene of interest is introduced into the phage genome as an in-frame fusion with the native coat protein gene. These vectors propagate independently as fully functional phage and display multiple copies of the foreign polypeptide. Phagemid vectors, in contrast, are plasmids that contain a phage origin of replication and packaging signal in addition to a bacterial origin of replication. Phagemids carry the gene of interest fused to a recombinant copy of the coat protein gene and, upon rescue with a helper phage, are packaged into progeny virus with the displayed polypeptide incorporated into the phage coat. The requirement for helper phage causes phagemid vectors to be more labor-intensive than phage vectors, and complicates efforts to quantitate the number of phage that are present in any given sample. Furthermore, since phage particles can draw upon both wild-type coat proteins and fusion coat proteins for assembly, some proportion of the resultant phage will not display the polypeptide sequence of interest, resulting in low display efficiency. Hybrid vectors resemble phage vectors in that the fusion protein is carried in the phage genome and no helper phage are needed, but they also resemble phagemid systems in that the genome also carries a wild-type copy of the fusion protein. Previous reports of pIX phage display describe fusions in the context of phagemid vectors; display of polypeptides on pIX from a hybrid or phage vector has not previously been reported. Display of polypeptides on pVII from a phage vector has recently been reported.

There is a need for providing synthetic non-antibody peptide or protein libraries and methods that simultaneously deliver the critical elements of human therapeutic peptides and proteins of high affinity and activity, high productivity, good solution properties, and a propensity of low immune response when administered in man. There is a further need to increase the efficiency of non-antibody peptide or protein isolation from synthetic libraries, relative to current methods, to reduce the resource costs of non-antibody peptide or protein discovery and accelerate delivery of non-antibody peptides or proteins for biological evaluation. The libraries and methods of this invention meet these needs by coupling comprehensive design, assembly technologies, and phage pIX Peptide or protein display.

### SUMMARY OF THE INVENTION

The present invention provides engineered pIX phage vectors that can be used with pVII and pIX phage display for generating peptide or protein libraries using pIX from M13 phage, e.g., using mutagenesis or other diversity producing techniques, optionally with in line maturation, to provide an efficient and fast platform for peptide or protein and non-antibody peptide or protein fragment generation and selection of therapeutic non-antibody peptides or proteins. According to the present invention, hybrid phage vectors are provides that have been engineered to include a second recombinant pIX coding region linked to an upstream signal peptide and inducible promoter.

The present invention provides a phage vector for the display of peptides and proteins as fusions to pIX or pVII phage proteins for use in expressing such peptides or proteins as peptide or protein libraries for use, e.g., but not limited to screening, selection, engineering, maturation or other uses, e.g., providing potential therapeutic or diagnostic peptides or proteins. Because the regulatory and coding regions of the native gene IX overlap those of pVII and pVIII, simple fusions to the terminus of this gene would likely inactivate the phage (Hill and Petersen, J. of Virol. 44:32-46, 1982). The current vector includes the fusion while at the same time preserves the regulatory region of the native coat proteins. The use of this vector, rather than the phagemid, obviates the need for helper phage and significantly reduces the amount of time and effort needed to culture the phage during selection and analysis. Furthermore, the multivalency nature of these display systems makes it easier to detect low affinity binders.

Thus the invention provides a novel vector construct for use in expressing peptides or proteins in a pIX phage display format for construction of polypeptide arrays. In particular, the invention describes an engineered pIX phage vector that includes a second recombinant pIX coding sequence encoding a fusion polypeptide, wherein the fusion polypeptide comprises an exogenous polypeptide fused to the amino terminus of a filamentous phage pVII or pIX protein. Preferably, the phage particle comprises the expressed fusion protein on the surface of the phage particle.

In aspect, the present invention provides an engineered recombinant nucleic acid phage vector for expressing phage display fusion peptides or proteins that bind to selected biologically active ligands, comprising (a) a recombinant phage leader coding nucleic acid sequence; operably linked to: (b) a recombinant tag, promoter, or selection coding nucleic acid sequence; operably linked to: (c) a recombinant pIX or pVII encoding nucleic acid sequence; operably linked to: (d) a recombinant restriction site; operably linked to: (e) a peptide linker encoding nucleic acid sequence; operably linked to a: (f) a first exogenous peptide or protein encoding sequence that selectively binds to a biologically active ligand; (g) a pVII encoding nucleic acid sequence; (h) a native pIX encoding nucleic acid sequence; (i) a pIII encoding nucleic acid sequence; and (j) a pVI encoding nucleic acid sequence.

Such an engineered nucleic acid phage vector can include wherein said phage leader coding sequence is a pelB sequence. Such an engineered nucleic acid phage vector can include wherein recombinant tag or selection sequence is a FLAG tag sequence. Such an engineered nucleic acid phage vector can include wherein recombinant tag or selection sequence is selected from SEQ ID NOS:3, 4, 5, or 6. Such an engineered nucleic acid phage vector can include wherein said FLAG tag sequence comprises SEQ ID NO:2. Such an engineered nucleic acid phage vector can include wherein said promoter is an inducible promoter. Such an engineered nucleic acid phage vector can include wherein said inducible promoter is a lac promoter. Such an engineered nucleic acid phage vector can include wherein said peptide linker is selected from SEQ ID NOS:7 and 8. Such an engineered nucleic acid phage vector can include wherein said exogenous first peptide or protein is a putative biologically active proteins or peptides. Such an engineered nucleic acid phage vector can include wherein said biologically active ligands mediate at least one biological in vivo activity. Such an engineered nucleic acid phage vector can include wherein said vector encodes a second exogenous peptide or protein fused to at least one phage coat protein.

The invention also includes a bacterial host cell comprising an engineered nucleic acid phage vector. The host cell can express a biologically active fusion protein.

The invention also relates to a biologically active fusion protein expressed by a bacterial host cell according to the invention. The invention also relates to a biologically active exogenous peptide or protein derived from said fusion protein.

The invention also relates to a phage library of bacterial host cells comprising a plurality of engineered nucleic acid phage vectors according to the present invention. The phage library can include wherein variants of said exogenous first peptides or proteins are expressed.

The invention also provides a method for screening a phage peptide or protein library for exogenous peptide or proteins having a desired biological activity, comprising (a) expressing exogenous peptides or proteins from a phage library, and (b) selecting bacterial cells expressing an exogenous peptide or protein having said desired biological activity. The invention also provides an exogenous peptide or protein encoding nucleic acid, obtained from such a method.

In one embodiment, the phage vector further encodes a second fusion polypeptide, wherein the second fusion polypeptide comprises a second exogenous polypeptide fused to the amino terminus of the pIX or pVII protein and the first exogenous polypeptide in the first fusion polypeptide is fused to the amino terminus of the pIX or pVII protein. In one embodiment, the first and second fusion polypeptides can associate to form a heterodimeric protein complex, such as a target protein, a receptor, a nucleic acid binding protein or an enzyme.

In another embodiment, the invention describes a vector for expressing a fusion protein on the surface of a filamentous phage comprising a cassette for expressing the fusion protein. The cassette includes upstream and downstream translatable DNA sequences operatively linked via a sequence of nucleotides adapted for directional ligation of an insert DNA, i.e., a polylinker, where the upstream sequence encodes a prokaryotic secretion signal, the downstream sequence encodes a pVII or pIX filamentous phage protein. The translatable DNA sequences are operatively linked to a set of DNA expression signals for expression of the translatable DNA sequences as portions of the fusion polypeptide. In a preferred variation, the vector optionally further comprises a second cassette for expressing a second fusion protein on the surface of the filamentous phage, wherein the second cassette has the structure of the first cassette with the proviso that the first fusion protein expression cassette encodes pIX or pVII protein and/or the second fusion protein expression cassette encodes pIX or pVII protein. The vector is used as a phage genome to express heterodimeric protein complexes on the surface of the phage particle in which the two exogenous polypeptides of the heterodimer are anchored on the phage particle by the fusion to the first and second phage proteins, pVII and/or pIX.

In another embodiment, the invention contemplates a library of phage particles according to the present invention, i.e., a combinatorial library, based on the engineered pIX phage vector in which representative particles in the library each display a different fusion protein. Where the particle displays a heterodimeric protein complex, the library comprises a combinatorial library of heterodimers, such as non-antibody peptides or proteins in the form of a library of Fv molecules. Preferred libraries have a combinatorial diversity of at least 10³, 10⁴, 10⁵, 10⁶, 10⁷, 10⁸, 10⁹, 10¹⁰, 10¹¹, 10¹², 10¹³, or any range or value therein, of fusion peptides or proteins.

A related embodiment describes a fusion protein comprising first and second polypeptides expressed from an engineered pIX phage vector of the invention, wherein the first polypeptide is an exogenous protein and the second polypeptide is a filamentous phage pVII or pIX protein, wherein the exogenous protein is fused to the amino terminus of the filamentous phage protein.

Still further, the invention contemplates a variety of methods for expressing proteins or peptides expressed from an engineered pIX phage vector of the invention, for producing a combinatorial library of phage, including by cloning repertoires of genes encoding an exogenous polypeptide into a vector of the present invention, modifying the structure of the exogenous polypeptides in a library by mutagenesis, by random combination of populations of first and second fusion protein libraries, by target and affinity selection (“panning”) to alter the diversity of a library, and the like.

The design of proteins with improved or novel functions is an important goal with a variety of medical, industrial, environmental, and basic research applications. Following the development of combinatorial non-antibody peptide or protein libraries using engineered pIX phage vectors, a powerful next step is the evolution toward artificial non-antibody peptide or protein constructs as well as other protein motifs in which dimeric species are native or might be functional.

The present invention addresses these challenges by providing a phage-display format using an engineered pIX phage vector for the construction of combinatorial polypeptide arrays in which pVII and/or pIX are utilized for the display of fusion proteins that express monomeric or dimeric peptide or protein species.

Inherent in the scope and power of the technology is the ability to display a variety of proteins that can engage in monomeric or dimeric interactions. These include not only non-antibody peptides or proteins, but also some enzymes, hormones and hormone receptors, and DNA-binding proteins. The display technology described herein can be used for combinatorial alteration of non-antibody peptide or protein framework regions and to reorganize and miniaturize the non-antibody peptide or protein structure or to display DNA binding proteins, such as repressors, as a library of heterodimers for selection against particular DNA sequences of clinical and therapeutic importance.

Thus the present technology provides for the display and selection of a library of peptides or proteins and combinatorial libraries in which members consist of monomeric, homodimeric or heterodimeric arrays.

It should be understood that both the foregoing general description and the following detailed description are exemplary and explanatory only and are not restrictive of the invention as claimed.

### DESCRIPTION OF THE FIGURES

FIG. 1A-D. Synthetic DNA insert for the expression of recombinant HA-pIX fusion protein in the M13-9 vector.

FIG. 2. Map of pMOM 60 showing the locations of native phage coat protein genes and the inserted recombinant pIX gene.

FIG. 3. Map of M13-9 showing the locations of native phage coat protein genes and the inserted pelB signal sequence and HA epitope.

FIG. 4A-E. ELISA showing specific binding of respective antibodies to HA-pIX fusion (a), FLAG-pIX fusion (b), His-6-pIX fusion (c), and specific binding of sEGFR-mimetibody to PHPEP 190-pIX (d) and EGF-pIX fusion (e) on M13-9 phage.

### DETAILED DESCRIPTION OF THE INVENTION

The present invention provides engineered pIX phage vectors that can be used with pVII and pIX phage display for generating peptide or protein libraries using pIX from M13 phage, e.g., using mutagenesis or other diversity producing techniques, optionally with in line maturation, to provide an efficient and fast platform for peptide or protein and non-antibody peptide or protein fragment generation and selection of therapeutic non-antibody peptides or proteins. According to the present invention, hybrid phage vectors are provided that have been engineered to include a second recombinant pIX coding region linked to an upstream signal peptide and inducible promoter.

The present invention provides a hybrid vector for the display of peptides and proteins as fusions to pIX or pVII phage proteins for use in expressing such peptides or proteins as peptide or protein libraries for use, e.g., but not limited to screening, selection, engineering, maturation or other uses, e.g., providing potential therapeutic or diagnostic peptides or proteins. Because the regulatory and coding regions of the native gene IX overlap those of pVII and pVIII, simple fusions to the terminus of this gene would likely inactivate the phage (Hill and Petersen, J. of Virol. 44:32-46, 1982) (8). Instead, a derivative of M13mp19 has been engineered to include a second recombinant pIX coding region linked to an upstream signal peptide and inducible promoter. The use of this vector, rather than a phagemid, obviates the need for helper phage and significantly reduces the amount of time and effort needed to culture the phage during selection and analysis. Furthermore, the number of phage grown with this vector can be determined more easily than the number of phage grown from a phagemid.

Thus the invention provides a novel vector construct for use in expressing peptides or proteins in a pIX phage display format for construction of polypeptide arrays. In particular, the invention describes an engineered pIX phage vector that includes a second recombinant pIX coding sequence encoding a fusion polypeptide, wherein the fusion polypeptide comprises an exogenous polypeptide fused to the amino terminus of a filamentous phage pVII or pIX protein. Preferably, the phage particle comprises the expressed fusion protein on the surface of the phage particle.

The human peptide or protein de novo library generated using such engineered pIX phage vectors described herein is distinct from current non-antibody peptide or protein library state-of-the-art by its displaying via pIX gene of M13 phage.

Filamentous Phage

The present invention contemplates using engineered pIX phage vectors as described herein with a pIX or pVII phage encoding at least one recombinant fusion peptide or protein. The fusion protein comprises an exogenous polypeptide portion fused to the amino terminus of a filamentous phage pVII or pIX protein.

By “exogenous” is meant that the polypeptide fused to the phage protein is not normally associated with the phage pVII or pIX protein in wild-type varieties of filamentous phage, but rather are foreign to the normal phage protein.

In a preferred embodiment, a filamentous phage encapsulates a genome which encodes a first and/or second fusion protein, where the first fusion protein comprises a first exogenous polypeptide fused to pVII or pIX and the second fusion protein comprises a second exogenous polypeptide fused to pIX or pIX.

The filamentous phage will further contain the fusion protein(s) displayed on the surface of the phage particle, as described herein. Thus, where there are at least first and second fusion proteins, the phage can display these proteins in a functional manner such that the first and second exogenous polypeptides can interact as a heterodimer to form a functional two-chain protein complex on the phage surface.

In a fusion protein present on a phage of this invention, the “fusion” between the exogenous polypeptide and the filamentous phage pVII or pIX protein can comprise a typical amide linkage, or can comprise a linker polypeptide (i.e., a “linker”) as described in the Examples. Any of a variety of linkers can be used which are typically a stretch of about 5 to 50 amino acids in length. Particularly preferred linkers provide a high degree of mobility to the fusion protein at the point of the linker.

Library design: prior synthetic libraries have incorporated some of the following, but none have included all in a comprehensive manner.

Position and nature of sequence diversity. Sequence diversity is a hallmark of how human proteins are provided endogenously that provide high-affinity, selective binding entities. This generation and accumulation of sequence diversity is not random. The site and type of nucleotide mutations of genomic sequences are biased by DNA sequence and mechanism but only mutations that provide binding and functional advantage are selected and stored, often along with neutral substitutions. While not amenable to prediction from mechanism, databases of known human peptide or protein sequences and structure-function analysis identifies positions and amino substitutions most frequently associated with recognition of a desired target or antigen, including differentiation between protein, peptide and small molecule antigens. The libraries of the present invention provide this natural human diversity by utilizing designed degenerate oligonucleotides to incorporate substitutions into putative binding regions and functional areas of the peptide or protein sequences that are expressed.

Expression, biochemical, and biophysical properties. Preferred human non-antibody peptides or proteins have desired biological and binding activities, but also are efficiently produced from a variety of hosts, are stable, and have good solution properties. High-frequency germline gene usage (1d) also indicates good expression in mammalian systems. In addition, non-antibody peptides or proteins recovered from libraries by bacterial phage display methods of selection or screening should be expressed well in the bacterial host. The libraries of the invention are based on human germline derived templates that are well-expressed and purified from standard recombinant mammalian hosts (e.g. HEK 293 and CHO cells) as well as bacterial hosts, and have high stability and good solution properties.

Library assembly technologies. Preferred de novo non-antibody peptide or protein libraries are of high diversity (>10¹⁰), amenable to alteration, and easy to assemble and have a low background of undesired sequences. These background sequences include parental template and low-targeted diversity. Coupling the following methods accelerates library assembly and leads to low background. (a) Kunkle-based single-stranded mutagenesis; (b) Palindromic loop with restriction site; (c) Megaprimer

pIX peptide or protein phage display. All prior filamentous de novo human non-antibody peptide or protein libraries utilize pIII or pVIII phage coat proteins for display. The combination of pIX with the selected Peptide or protein templates is a more efficient selection system for recovering non-antibody peptides or proteins that retain their selected properties upon conversion into mAbs and other related molecules.

Peptide or protein display. Peptide or proteins are natural segments of human non-antibody peptides or proteins and they better recapitulate their activity when engineered into full non-antibody peptides or proteins. Efficient filamentous display of peptide or proteins can require properties beyond good expression in the bacterial host. Peptide sequences used on libraries of present invention were chosen for efficient display by pIX on filamentous phage.

Phagemid display. The peptide or protein molecule may be large relative to the phage pIX coat protein and thus can interfere with assembly of recombinant phage particles if linked to all pIX proteins produced in the bacterial cell. One approach to by-pass this interference is to use a pIX phagemid system, whereby both wild-type and peptide or protein-linked pIX proteins can be incorporated into the recombinant phage particle. In a preferred application, libraries of the present invention are displayed by pIX in a phagmid system.

Phage coat protein pIX for display. Like pIII, pIX is present at low copy number on the phage and is amenable to affinity selection of displayed peptide or proteins. However, the pIII protein is critically involved in the infection process and proteins displayed on this protein can interfere with the efficiency of infection. Moreover, either the heavy chain Fd or light chain segments can be fused to pIX for display. The libraries of the present invention displayed on the pIX protein are predicted to be efficiently replicated and presented for selection and/or screening.

Peptide or protein-pIX expression. One approach to screening peptide or proteins recovered from phage libraries is to remove the phage coat protein that is linked to the peptide or protein molecule for display. The small size of the pIX protein provides the option of production of screening of peptide or proteins directly without this step.

Phage construction. Suitable M13 or similar types of phage vectors can be used as engineered according to the present invention. Such vectors that encode pIX or pVII fusion proteins with suitable regulatory, selection, restriction and other needed sites and sequences (e.g., promoters, signal sequences, leaders (e.g., pelB), ribosome binding sites (e.g., Shine-Delgano), tags (e.g., FLAG tag); transcriptional terminator (e.g., trpA), selection (e.g., LACZ), restriction sites (e.g., HindIII, EcoRI), peptide linkers, and the like) are modified according to known techniques to also include a second pIX and/or pVII coding sequence linked to an upstream signal peptide encoding sequence and an inducible promoter (e.g., LacZa). This engineering obviates the need for a helper phage and also signigicantly reduces the time and effort needed to culture or grow the phage during the selection and/or analysis steps. Additionally, the number of phage needed to be groan can be determined more easily than using other vectors.

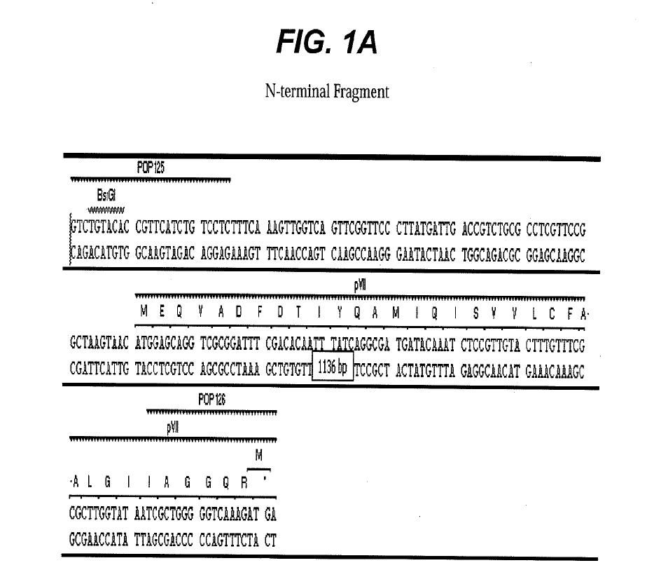

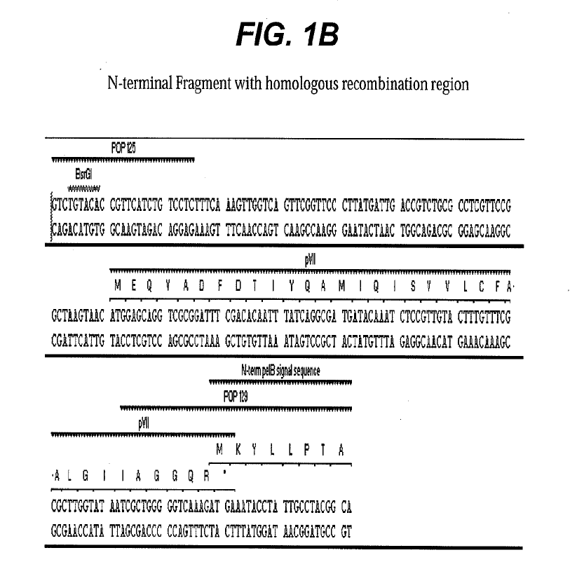

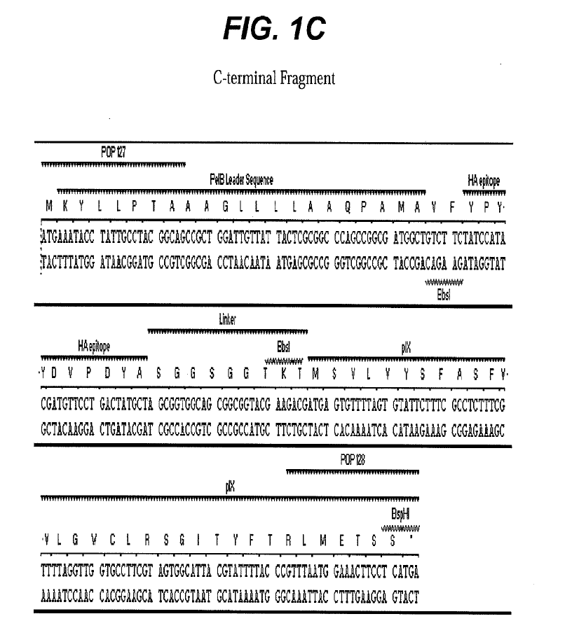

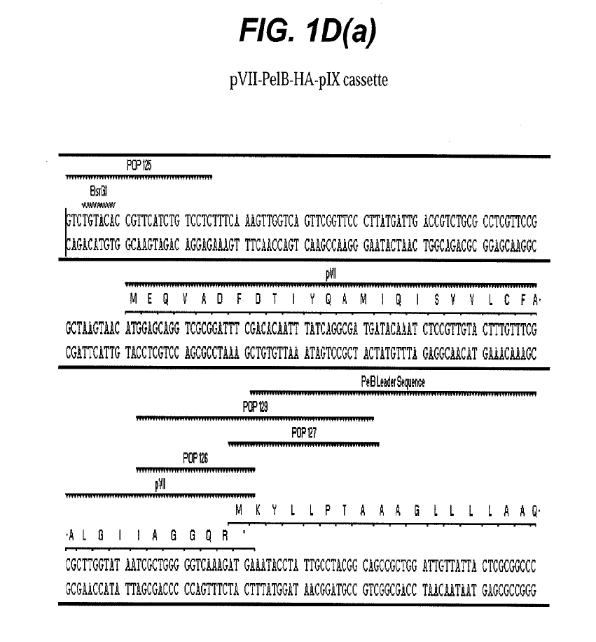

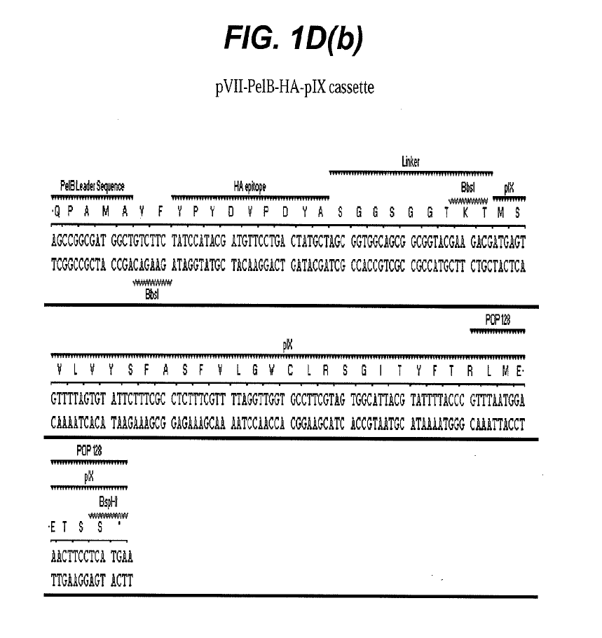

As a non-limiting example, M13KE, a derivative of M13mp19, are known phage vectors that can be used to provide an engineered pIX phage vector of the present invention by inserting a recombinant pIX gene. The recombinant region can be inserted, e.g., into the lacZa region of M13mp19, in the intergenic region of the phage genome, and thus the lac promoter drives transcription of the recombinant gene IX fusion. The insert (FIG. 1) can include a Shine-Delgarno sequence (ribosome-binding site), a signal sequence from pectate lyase B (pelB), dual BbsI restriction enzyme recognition sites for future cloning, the pIX coding region, and the trpA transcriptional terminator. A FLAG tag peptide DYKDDDDK and a five-amino acid linker (M13-99: GGTKT) or a nine-amino acid linker (M13-99L: SGGSGGTKT) included between pelB and gene IX.

Additional peptides (e.g, but not limited to those in Table 1) with various lengths and charges can be displayed on the amino terminus of pIX with the nine-amino acid linker to determine which linker are most suitable for expressing a particular polypeptide. In addition, one or more exogenous fusion peptides are displayed on pIX or pVII.

The final phage vector can be analyzed containing the recombinant pIX genes for display of peptide tags, e.g., in ELISA experiments. Phages that bound to immobilized target peptides or proteins can be detected with an anti-M13/target conjugate or any other detection of the express exogenous peptide.

Advantages. The phage system for display of peptides and proteins on pIX offers advantages of avidity, speed, and convenience over the previously developed phagemid system. Due to the avidity effect, the binding signal is significantly increased when compared with either a phagemid or a hybrid system. Therefore it would serve as an ideal system to identify weak binders from a large pool. Such an amplification of the binding signal can be crucial for peptides, which tend to have intrinsically weak affinity without affinity maturation. Peptides with different length, charge, linear, and cyclic and a small globular protein have been successfully displayed on pIX and are disclosed here. The time saving for the phage vectors is significant as well. Phage can be infected into host cells and amplified in an afternoon, essentially in a single step. Amplification of phagemid, by contrast, requires infection and outgrowth of the phagemid, superinfection with helper phage at a defined culture density, and amplification of rescued phage. The procedure thus entails additional steps and operator input and—at a minimum—an overnight culture. Over the course of the repeated selection cycles and multiple rounds of screening involved in a typical selection experiment, the time savings of a phage system can be significant. In addition, a phage system abolishes the need for helper phage infection only one type of phage genome is present that can be packaged into a phage particle. This generates a homogeneous population of phage allowing a precise measurement of viral particles containing the fusion genome.

While having described the invention in general terms, the embodiments of the invention will be further disclosed in the following examples that should not be construed as limiting the scope of the claims.

### Example 1

### Exemplary Engineered Phage Vector Construction

Type-9 phage vector construction: A prototype M13-9 vector, PHPEP208 was constructed that contains a signal sequence from pectate lyase B (pelB) and dual BbsI restriction enzyme recognition sites for future cloning inserted between pVII and pIX genes in the phage genome M13KE, a derivative of M13mp19. In the unmodified M13KE phage genome, the terminal nucleotide base of the last amino acid codon for pVII gene is the first nucleotide base of ATG start codon for pIX. This last and the first nucleotide sharing between the pVII and the pIX gene was preserved in PHPEP 208 between the pVII gene and ATG start codon for the pelB signal sequence. An influenza hemagglutin (HA) peptide YPYDVPDYA and a nine-amino acid linker SGGSGGTKT were included between pelB signal sequence and gene pIX. Three other peptides (Table 1) with various lengths and charges and a small globular protein, epidermal growth factor (EGF) (SEQ ID NO:6), were subcloned into PHPEP208 and displayed on the amino terminus of pIX with the nine amino acid linker.

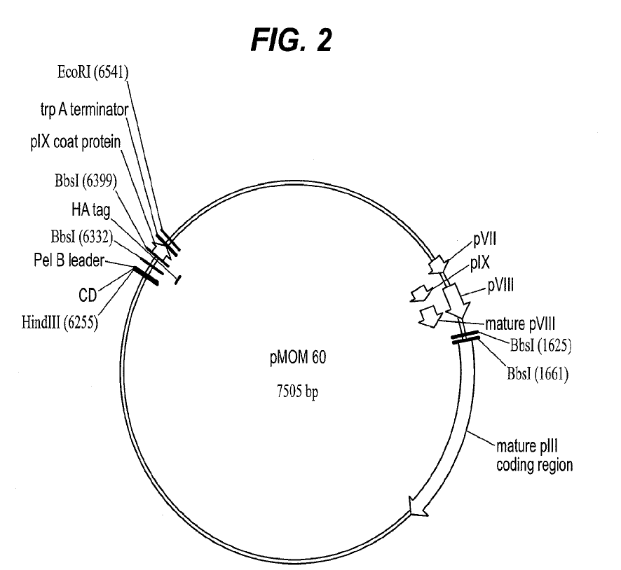

Methods. DNA encoding the pVII-PelB-HA-pIX cassette (FIG. 1d), flanked by BsrGI and BspHI enzyme recognition sites, was generated by two series of PCR amplifications from the M13-99 phage genome containing HA cassette (MOM 60) to obtain N-terminal and C-terminal fragment. Then, two fragments were joined together by an overlapping PCR recombination reaction. MOM 60 contains a recombinant pIX gene inserted into the phage genome M13KE, a derivative of M13mp19 (FIG. 2). The recombinant region has been inserted into the lacZa region of M13mp19, in the intergenic region of the phage genome, and thus the lac promoter drives transcription of the recombinant gene IX fusion.

The insert included a Shine-Delgarno sequence (ribosome-binding site), a signal sequence from pectate lyase B (pelB), dual BbsI restriction enzyme recognition sites for future cloning, the pIX coding region, and the trpA transcriptional terminator. A HA peptide YPYDVPDYA (SEQ ID NO:2) and a nine-amino acid linker SGGSGGTKT (SEQ ID NO:7) was included between pelB and gene IX. To generate the N-terminal fragment, a stretch of DNA including BsrGI site and pVII gene was PCR amplified from MOM60 genome (FIG. 1a). Then a part of pelB signal sequence was added to its C-terminal end by PCR amplification to provide a 18-bp complementary base-paring site for future recombination reaction with the C-terminal fragment (FIG. 1b). The C-terminal fragment was generated by PCR amplification of a DNA stretch containing pelB signal sequence, HA epitope, and the recombinant copy of pIX gene from HA cassette of MOM60 phage genome (FIG. 1c). The reverse oligo nucleotide primer contained the BspHI restriction site.

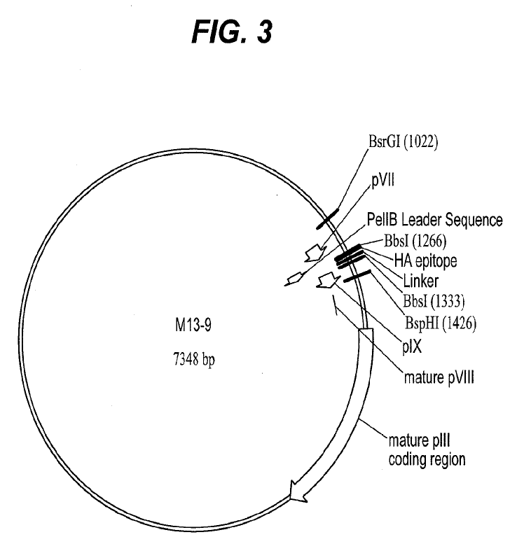

The N and C-terminal PCR fragments were allowed to anneal together at the complementary region and amplified by PCR (FIG. 1d). The cassette was restriction digested with BsrGI and BspHI enzymes and ligated into M13KE RF DNA that had been digested with BsrGI and BspHI. The final phage vector, M13-9 (SEQ ID NO. 1), is diagrammed in FIG. 3. For the other peptides, complimentary oligos were annealed together to generate appropriate DNA sequences. Annealed oligos contained compatible overhangs corresponding to the BbsI-digested M13-9 vector allowing for ligation of the peptide tag DNA and the vector. PHPEP 190 is a peptide that has affinity to soluble EGFR receptor. Eight of its amino acid residues are in a loop constrained by a disulfide bond. EGF was PCR amplified, digested with BbsI restriction endonuclease, and ligated into the BbsI digested M13-9. Recombinant phages were plated to isolate single plaques on a lawn of XL-1 Blue host E. coli cells (Stratagene). Phage plaques were resuspended in media, and the phages were allowed to diffuse from the agar. The phages were infected into XL-1 Blue and cultured for 4.5 hours at 37° C. After phage growth and induction, bacteria were removed by centrifugation, and the phage were precipitated from the culture supernatant with 4% PEG-8000, 0.5 M NaCl and incubation at 4° C. overnight. The phage particles were recovered by centrifugation, and the phage pellet was resuspended in phosphate-buffered saline (PBS).

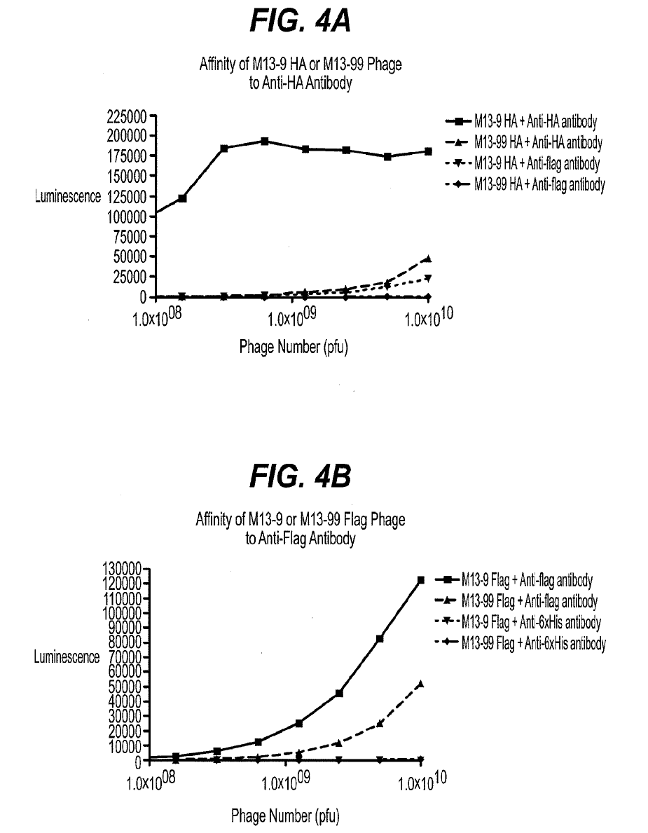

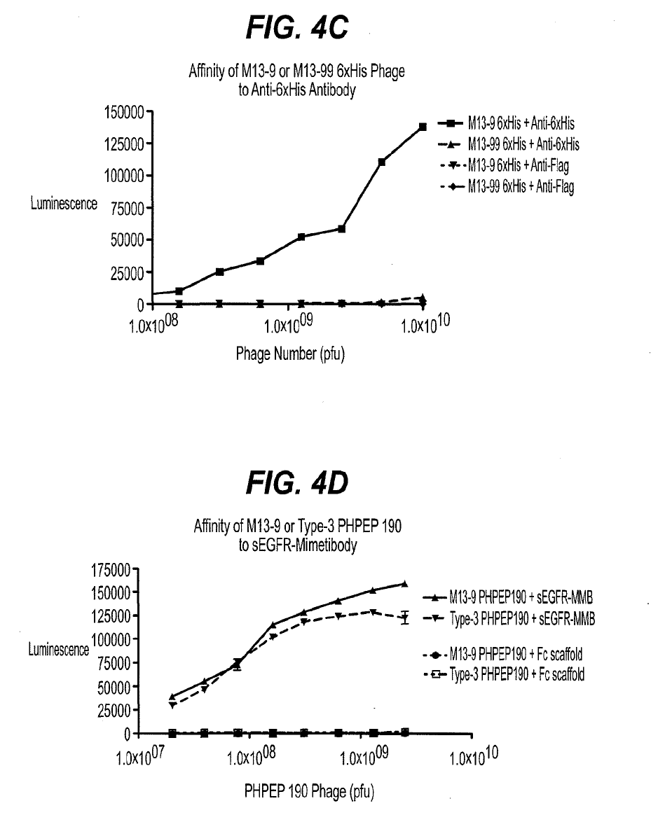

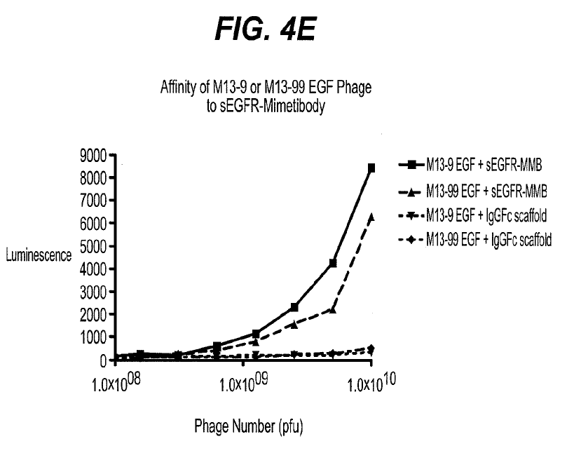

Analysis of the displayed peptides. The phages containing the peptides or the protein were tested for display in ELISA experiments. Phages that bound to immobilized monoclonal antibodies or soluble EGFR-mimetibody were detected with an anti-M13/HRP conjugate. M13-9 with the HA insert was seen to bind specifically to the anti-HA antibody versus the anti-flag antibody, indicating successful display of the HA tag on the recombinant pIX (FIG. 4a). The ELISA data for the other peptides and EGF are shown in FIGS. 4b, 4c, 4d, and 4e. Anti-his and anti-flag antibodies served as targets for the appropriate phages. Soluble EGFR-mimetibody was used to test for the display of PHPEP 190 peptides and EGF protein. Human IgG1 Fc scaffold was used as the negative control for PHPEP 190 and EGF phage ELISA. These results indicate that these peptides and the EGF protein were also successfully displayed on the recombinant pIX.

Methods. Wells of a Maxisorp ELISA plate (NUNC) were coated with 500 ng of monoclonal antibodies or soluble EGFR-mimetibody at 5 μg/ml in PBS, overnight at 4° C. The wells were rinsed twice with Tris-buffered saline containing 0.1% Tween-20 (TBS-T) and blocked with Starting Block (Pierce) for 1 hr at room temperature. The wells were rinsed again. PEG-precipitated phage (10⁸˜10¹⁰ pfu), diluted in Starting Block, were added to the wells and incubated for 1 hr at room temperature with shaking. The wells were rinsed thrice with TBS-T, and anti-M13/horseradish peroxidase conjugate (GE Healthcare), diluted 1:5000 in Starting Block, was added to the wells and incubated for 1 hr at room temperature with shaking. The wells were rinsed thrice with PBS-T, and POD chemiluminescent substrate (Roche) was added and detected on a Tecan plate reader.

TABLE 1
Peptide sequences cloned into the pIX hybrid
expression vector.

| Peptide | |
|---|---|
| tags | Amino acid sequence |
| FLAG | DYKDDDDK |
| | (SEQ ID NO: 3) |
| HA | YPYDVPDYA |
| | (SEQ ID NO: 2) |
| HIS | HHHHHH |
| | (SEQ ID NO: 4) |
| PHPEP | GGDPCTWEVWGRECLQGG |
| 190 | (SEQ ID NO: 5) |
| EGF | MAVFNSDSECPLSHDGYCLHDGVCMYIEALDKYACNCVVGYIGER |
| | CQYRDLKWWELR |
| | (SEQ ID NO: 6) |

### REFERENCES

1. Kehoe, J. W., and B. K. Kay. 2005. Filamentous phage display in the new millennium. Chem Rev 105:4056.
2. Iannolo, G., O. Minenkova, R. Petruzzelli, and G. Cesareni. 1995. Modifying filamentous phage capsid: limits in the size of the major capsid protein. J Mol Biol 248:835.
3. Gao, C., S. Mao, C. H. Lo, P. Wirsching, R. A. Lerner, and K. D. Janda. 1999. Making artificial antibodies: a format for phage display of combinatorial heterodimeric arrays. Proc Natl Acad Sci USA 96:6025.
4. Gao, C., S. Mao, G. Kaufmann, P. Wirsching, R. A. Lerner, and K. D. Janda. 2002. A method for the generation of combinatorial antibody libraries using pIX phage display. Proc Natl Acad Sci USA 99:12612.
5. Gao, C., S. Mao, H. J. Ditzel, L. Farnaes, P. Wirsching, R. A. Lerner, and K. D. Janda. 2002. A cell-penetrating peptide from a novel pVII-pIX phage-displayed random peptide library. Bioorg Med Chem 10:4057.
6. Endemann, H., and P. Model. 1995. Location of filamentous phage minor coat proteins in phage and in infected cells. J Mol Biol 250:496.
7. Hill, D. F., and G. B. Petersen. 1982. Nucleotide Sequence of Bacteriophage f1 DNA. Journal of Virology 44:32.

### SEQUENCE LISTING

| SEQ ID NO: 1 | |
|---|---|
| Sequence of M13-9 (HA) | |
| aatgctacta ctattagtag aattgatgcc accttttcag ctcgcgcccc aaatgaaaat | 60 |
| atagctaaac aggttattga ccatttgcga aatgtatcta atggtcaaac taaatctact | 120 |
| cgttcgcaga attgggaatc aactgttaca tggaatgaaa cttccagaca ccgtacttta | 180 |
| gttgcatatt taaaacatgt tgagctacag caccagattc agcaattaag ctctaagcca | 240 |
| tccgcaaaaa tgacctctta tcaaaaggag caattaaagg tactctctaa tcctgacctg | 300 |
| ttggagtttg cttccggtct ggttcgcttt gaagctcgaa ttaaaacgcg atatttgaag | 360 |
| tctttcgggc ttcctcttaa tctttttgat gcaatccgct ttgcttctga ctataatagt | 420 |
| cagggtaaag acctgatttt tgatttatgg tcattctcgt tttctgaact gtttaaagca | 480 |
| tttgaggggg attcaatgaa tatttatgac gattccgcag tattggacgc tatccagtct | 540 |
| aaacatttta ctattacccc ctctggcaaa acttcttttg caaaagcctc tcgctatttt | 600 |
| ggtttttatc gtcgtctggt aaacgagggt tatgatagtg ttgctcttac tatgcctcgt | 660 |
| aattcctttt ggcgttatgt atctgcatta gttgaatgtg gtattcctaa atctcaactg | 720 |
| atgaatcttt ctacctgtaa taatgttgtt ccgttagttc gttttattaa cgtagattt | 780 |
| tcttcccaac gtcctgactg gtataatgag ccagttctta aaatcgcata aggtaatta | 840 |
| caatgattaa agttgaaatt aaaccatctc aagcccaatt tactactcgt tctggtgtt | 900 |
| ctcgtcaggg caagccttat tcactgaatg agcagctttg ttacgttgatttgggtaatg | 960 |
| aatatccggt tcttgtcaag attactcttg atgaaggtca gccagcctat gcgcctggtc | 1020 |
| tgtacaccgt tcatctgtcc tctttcaaag ttggtcagtt cggttccctt atgattgacc | 1080 |
| gtctgcgcct cgttccggct aagtaacatg gagcaggtcg cggatttcga cacaatttat | 1140 |
| caggcgatga tacaaatctc cgttgtactt tgtttcgcgc ttggtataat cgctgggggt | 1200 |
| caaagatgaa atacctattg cctacggcag ccgctggatt gttattactc gcggcccagc | 1260 |
| cggcgatggc tgtcttctat ccatacgatg ttcctgacta tgctagcggt ggcagcggcg | 1320 |
| gtacgaagac gatgagtgtt ttagtgtatt ctttcgcctc tttcgtttta ggttggtgcc | 1380 |
| ttcgtagtgg cattacgtat tttacccgtt taatggaaac ttcctcatga aaaagtcttt | 1440 |
| agtcctcaaa gcctctgtag ccgttgctac cctcgttccg atgctgtctt tcgctgctga | 1500 |
| gggtgacgat cccgcaaaag cggcctttaa ctccctgcaa gcctcagcga ccgaatatat | 1560 |
| cggttatgcg tgggcgatgg ttgttgtcat tgtcggcgca actatcggta tcaagctgtt | 1620 |
| taagaaattc acctcgaaag caagctgata aaccgataca attaaaggct ccttttggag | 1680 |
| cctttttttt ggagattttc aacgtgaaaa aattattatt cgcaattcct ttagtggtac | 1740 |
| ctttctattc tcactcggcc gaaactgttg aaagttgttt agcaaaatcc catacagaaa | 1800 |
| attcatttac taacgtctgg aaagacgaca aaactttaga tcgttacgct aactatgagg | 1860 |
| gttgtctgtg gaatgctaca ggcgttgtag tttgtactgg tgacgaaact cagtgttacg | 1920 |
| gtacatgggt tcctattggg cttgctatcc ctgaaaatga gggtggtggc tctgagggtg | 1980 |
| gcggttctga gggtggcggt tctgagggtg gcggtactaa acctcctgag tacggtgata | 2040 |
| cacctattcc gggctatact tatatcaacc ctctcgacgg cacttatccg cctggtactg | 2100 |
| agcaaaaccc cgctaatcct aatccttctc ttgaggagtc tcagcctctt aatactttca | 2160 |
| tgtttcagaa taataggttc cgaaataggc agggggcatt aactgtttat acgggcactg | 2220 |
| ttactcaagg cactgacccc gttaaaactt attaccagta cactcctgta tcatcaaaag | 2280 |
| ccatgtatga cgcttactgg aacggtaaat tcagagactg cgctttccat tctggcttta | 2340 |
| atgaagatcc attcgtttgt gaatatcaag gccaatcgtc tgacctgcct caacctcctg | 2400 |
| tcaatgctgg cggcggctct ggtggtggtt ctggtggcgg ctctgagggt ggtggctctg | 2460 |
| agggtggcgg ttctgagggt ggcggctctg agggaggcgg ttccggtggt ggctctggtt | 2520 |
| ccggtgattt tgattatgaa aagatggcaa acgctaataa gggggctatg accgaaaatg | 2580 |
| ccgatgaaaa cgcgctacag tctgacgcta aaggcaaact tgattctgtc gctactgatt | 2640 |
| acggtgctgc tatcgatggt ttcattggtg acgtttccgg ccttgctaat ggtaatggtg | 2700 |
| ctactggtga ttttgctggc tctaattccc aaatggctca agtcggtgac ggtgataatt | 2760 |
| cacctttaat gaataatttc cgtcaatatt taccttccct ccctcaatcg gttgaatgtc | 2820 |
| gcccttttgt ctttagcgct ggtaaaccat atgaattttc tattgattgt gacaaaataa | 2880 |
| acttattccg tggtgtcttt gcgtttcttt tatatgttgc cacctttatg tatgtatttt | 2940 |
| ctacgtttgc taacatactg cgtaataagg agtcttaatc atgccagttc ttttgggtat | 3000 |
| tccgttatta ttgcgtttcc tcggtttcct tctggtaact ttgttcggct atctgcttac | 3060 |
| ttttcttaaa aagggcttcg gtaagatagc tattgctatt tcattgtttc ttgctcttat | 3120 |
| tattgggctt aactcaattc ttgtgggtta tctctctgat attagcgctc aattaccctc | 3180 |
| tgactttgtt cagggtgttc agttaattct cccgtctaat gcgcttccct gtttttatgt | 3240 |
| tattctctct gtaaaggctg ctattttcat ttttgacgtt aaacaaaaaa tcgtttctta | 3300 |
| tttggattgg gataaataat atggctgttt attttgtaac tggcaaatta ggctctggaa | 3360 |
| agacgctcgt tagcgttggt aagattcagg ataaaattgt agctgggtgc aaaatagcaa | 3420 |
| ctaatcttga tttaaggctt caaaacctcc cgcaagtcgg gaggttcgct aaaacgcctc | 3480 |
| gcgttcttag aataccggat aagccttcta tatctgattt gcttgctatt gggcgcggta | 3540 |
| atgattccta cgatgaaaat aaaaacggct tgcttgttct cgatgagtgc ggtacttggt | 3600 |
| ttaatacccg ttcttggaat gataaggaaa gacagccgat tattgattgg tttctacatg | 3660 |
| ctcgtaaatt aggatgggat attatttttc ttgttcagga cttatctatt gttgataaac | 3720 |
| aggcgcgttc tgcattagct gaacatgttg tttattgtcg tcgtctggac agaattactt | 3780 |
| taccttttgt cggtacttta tattctctta ttactggctc gaaaatgcct ctgcctaaat | 3840 |
| tacatgttgg cgttgttaaa tatggcgatt ctcaattaag ccctactgtt gagcgttggc | 3900 |
| tttatactgg taagaatttg tataacgcat atgatactaa acaggctttt tctagtaatt | 3960 |
| atgattccgg tgtttattct tatttaacgc cttatttatc acacggtcgg tatttcaaac | 4020 |
| cattaaattt aggtcagaag atgaaattaa ctaaaatata tttgaaaaag ttttctcgcg | 4080 |
| ttctttgtct tgcgattgga tttgcatcag catttacata tagttatata acccaaccta | 4140 |
| agccggaggt taaaaaggta gtctctcaga cctatgattt tgataaattc actattgact | 4200 |
| cttctcagcg tcttaatcta agctatcgct atgttttcaa ggattctaag ggaaaattaa | 4260 |
| ttaatagcga cgatttacag aagcaaggtt attcactcac atatattgat ttatgtactg | 4320 |
| tttccattaa aaaaggtaat tcaaatgaaa ttgttaaatg taattaattt tgttttcttg | 4380 |
| atgtttgttt catcatcttc ttttgctcag gtaattgaaa tgaataattc gcctctgcgc | 4440 |
| gattttgtaa cttggtattc aaagcaatca ggcgaatccg ttattgtttc tcccgatgta | 4500 |
| aaaggtactg ttactgtata ttcatctgac gttaaacctg aaaatctacg caatttcttt | 4560 |
| atttctgttt tacgtgctaa taattttgat atggttggtt caattccttc cataattcag | 4620 |
| aagtataatc caaacaatca ggattatatt gatgaattgc catcatctga taatcaggaa | 4680 |
| tatgatgata attccgctcc ttctggtggt ttctttgttc cgcaaaatga taatgttact | 4740 |
| caaactttta aaattaataa cgttcgggca aaggatttaa tacgagttgt cgaattgttt | 4800 |
| gtaaagtcta atacttctaa atcctcaaat gtattatcta ttgacggctc taatctatta | 4860 |
| gttgttagtg cacctaaaga tattttagat aaccttcctc aattcctttc tactgttgat | 4920 |
| ttgccaactg accagatatt gattgagggt ttgatatttg aggttcagca aggtgatgct | 4980 |
| ttagattttt catttgctgc tggctctcag cgtggcactg ttgcaggcgg tgttaatact | 5040 |
| gaccgcctca cctctgtttt atcttctgct ggtggttcgt tcggtatttt taatggcgat | 5100 |
| gttttagggc tatcagttcg cgcattaaag actaatagcc attcaaaaat attgtctgtg | 5160 |
| ccacgtattc ttacgctttc aggtcagaag ggttctatct ctgttggcca gaatgtccct | 5220 |
| tttattactg gtcgtgtgac tggtgaatct gccaatgtaa ataatccatt tcagacgatt | 5280 |
| gagcgtcaaa atgtaggtat ttccatgagc gtttttcctg ttgcaatggc tggcggtaat | 5340 |
| attgttctgg atattaccag caaggccgat agtttgagtt cttctactca ggcaagtgat | 5400 |
| gttattacta atcaaagaag tattgctaca acggttaatt tgcgtgatgg acagactctt | 5460 |
| ttactcggtg gcctcactga ttataaaaac acttctcaag attctggcgt accgttcctg | 5520 |
| tctaaaatcc ctttaatcgg cctcctgttt agctcccgct ctgattccaa cgaggaaagc | 5580 |
| acgttatacg tgctcgtcaa agcaaccata gtacgcgccc tgtagcggcg cattaagcgc | 5640 |
| ggcgggtgtg gtggttacgc gcagcgtgac cgctacactt gccagcgccc tagcgcccgc | 5700 |
| tcctttcgct ttcttccctt cctttctcgc cacgttcgcc ggctttcccc gtcaagctct | 5760 |
| aaatcggggg ctccctttag ggttccgatt tagtgcttta cggcacctcg accccaaaaa | 5820 |
| acttgatttg ggtgatggtt cacgtagtgg gccatcgccc tgatagacgg tttttcgccc | 5880 |
| tttgacgttg gagtccacgt tctttaatag tggactcttg ttccaaactg gaacaacact | 5940 |
| caaccctatc tcgggctatt cttttgattt ataagggatt ttgccgattt cggaaccacc | 6000 |
| atcaaacagg attttcgcct gctggggcaa accagcgtgg accgcttgct gcaactctct | 6060 |
| cagggccagg cggtgaaggg caatcagctg ttgcccgtct cgctggtgaa aagaaaaacc | 6120 |
| accctggcgc ccaatacgca aaccgcctct ccccgcgcgt tggccgattc attaatgcag | 6180 |
| ctggcacgac aggtttcccg actggaaagc gggcagtgag cgcaacgcaa ttaatgtgag | 6240 |
| ttagctcact cattaggcac cccaggcttt acactttatg cttccggctc gtatgttgtg | 6300 |
| tggaattgtg agcggataac aatttcacac aggaaacagc tatgaccatg attacgccaa | 6360 |
| gcttgcatgc ctgcaggtcc tcgaattcac tggccgtcgt tttacaacgt cgtgactggg | 6420 |
| aaaaccctgg cgttacccaa cttaatcgcc ttgcagcaca tccccctttc gccagctggc | 6480 |
| gtaatagcga agaggcccgc accgatcgcc cttcccaaca gttgcgcagc ctgaatggcg | 6540 |
| aatggcgctt tgcctggttt ccggcaccag aagcggtgcc ggaaagctgg ctggagtgcg | 6600 |
| atcttcctga ggccgatacg gtcgtcgtcc cctcaaactg gcagatgcac ggttacgatg | 6660 |
| cgcccatcta caccaacgta acctatccca ttacggtcaa tccgccgttt gttcccacgg | 6720 |
| agaatccgac gggttgttac tcgctcacat ttaatgttga tgaaagctgg ctacaggaag | 6780 |
| gccagacgcg aattattttt gatggcgttc ctattggtta aaaaatgagc tgatttaaca | 6840 |
| aaaatttaac gcgaatttta acaaaatatt aacgtttaca atttaaatat ttgcttatac | 6900 |
| aatcttcctg tttttggggc ttttctgatt atcaaccggg gtacatatga ttgacatgct | 6960 |
| agttttacga ttaccgttca tcgattctct tgtttgctcc agactctcag gcaatgacct | 7020 |
| gatagccttt gtagatctct caaaaatagc taccctctcc ggcattaatt tatcagctag | 7080 |
| aacggttgaa tatcatattg atggtgattt gactgtctcc ggcctttctc acccttttga | 7140 |
| atctttacct acacattact caggcattgc atttaaaata tatgagggtt ctaaaaattt | 7200 |
| ttatccttgc gttgaaataa aggcttctcc cgcaaaagta ttacagggtc ataatgtttt | 7260 |
| tggtacaacc gatttagctt tatgctctga ggctttattg cttaattttg ctaattcttt | 7320 |
| gccttgcctg tatgatttat tggatgtt | 7348 |
| (SEQ ID NO: 2) | |
| YPYDVPDYA | |
| (SEQ ID NO: 3) | |
| DYKDDDDK | |
| (SEQ ID NO: 4) | |
| HHHHHH | |
| (SEQ ID NO: 5) | |
| GGDPCTWEVWGRECLQGG | |
| (SEQ ID NO: 6) | |
| MAVFNSDSECPLSHDGYCLHDGVCMYIEALDKYACNCVVGYIGERCQYRDLKWWELR | |

1. An engineered recombinant nucleic acid phage vector for expressing phage display fusion peptides or proteins that bind to selected biologically active ligands, comprising
a. a recombinant pVII phage coding nucleic acid sequence; operably linked to:
b. a recombinant phage leader coding nucleic acid sequence; operably linked to:
c. a recombinant restriction site; operably linked to:
d. a peptide linker encoding nucleic acid sequence; operably linked to a:
e. a first exogenous peptide or protein encoding sequence that selectively binds to a biologically active ligand;
f. a native pIX encoding nucleic acid sequence;
g. a mature pVIII encoding nucleic acid sequence; and
h. a mature pIII encoding nucleic acid sequence

2. An engineered nucleic acid phage vector according to claim 1, wherein said phage leader coding sequence is a pelB sequence.

3. An engineered nucleic acid phage vector according to claim 1, wherein recombinant tag or selection sequence is an HA tag sequence.

4. An engineered nucleic acid phage vector according to claim 1, wherein recombinant tag or selection sequence is selected from SEQ ID NOS:3, 4.

5. An engineered nucleic acid phage vector according to claim 1, wherein said peptide linker is selected from SEQ ID NOS:6, 7 and 8.

6. An engineered nucleic acid phage vector according to claim 1, wherein said exogenous first peptide or protein is a putative biologically active proteins or peptides.

7. An engineered nucleic acid phage vector according to claim 1, wherein said biologically active ligands mediate at least one biological in vivo activity.

8. An engineered nucleic acid phage vector according to claim 1, wherein said vector encodes a second exogenous peptide or protein fused to at least one phage coat protein.

9. A bacterial host cell comprising an engineered nucleic acid phage vector according to claim 1.

10. A biologically active fusion protein expressed by a bacterial host cell according to claim 9.

11. A biologically active exogenous peptide or protein derived from said fusion protein according to claim 10.

12. A phage library of bacterial host cells comprising a plurality of engineered nucleic acid phage vectors according to claim 1.

13. A phage library according to claim 12, wherein variants of said exogenous first peptides or proteins are expressed.

14. A method for screening a phage peptide or protein library for exogenous peptide or proteins having a desired biological activity, comprising (a) expressing exogenous peptides or proteins from a phage library according to claim 13, and (b) selecting bacterial cells expressing an exogenous peptide or protein having said desired biological activity.

15. An exogenous peptide or protein encoding nucleic acid, obtained from a method according to claim 14.


SEQUENCE LISTING (7 sequences)

SEQ ID NO: 1 - DNA, 7345 bp, Homo Sapien
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagattttcttcccaacgtcctgactggtataatgagccagttcttaaaatcgcataaggtaattacaatgattaaagttgaaattaaaccatctcaagcccaatttactactcgttctggtgttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgaaatacctattgcctacggcagccgctggattgttattactcgcggcccagccggcgatggctgtcttctatccatacgatgttcctgactatgctagcggtggcagcggcggtacgaagacgatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagtggtacctttctattctcactcggccgaaactgttgaaagttgtttagcaaaatcccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaagatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcgctggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatacggtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 2 - protein, 9 aa, Homo Sapien
YPYDVPDYA

SEQ ID NO: 3 - protein, 8 aa, Homo Sapien
DYKDDDDK

SEQ ID NO: 4 - protein, 6 aa, Homo Sapien
HHHHHH

SEQ ID NO: 5 - protein, 18 aa, Homo Sapien
GGDPCTWEVWGRECLQGG

SEQ ID NO: 6 - protein, 57 aa, Homo Sapien
MAVFNSDSECPLSHDGYCLHDGVCMYIEALDKYACNCVVGYIGERCQYRDLKWWELR

SEQ ID NO: 7 - protein, 9 aa, Homo Sapien
SGGSGGTKT